(19) United States
(12) Patent Application Publication
(10) Pub. No.: US 2010/0175160 A1
(43) Pub. Date: Jul. 15, 2010

(54) VISOR AND METHOD OF MAKING THE SAME

(75) Inventor: Derek Leslie ARNOLD, Emmeloord (NL)

Correspondence Address: HOWREY LLP-EU, C/O IP DOCKETING DEPARTMENT, 2941 FAIRVIEW PARK DR., SUITE 200, FALLS CHURCH, VA, 22042, US

(73) Assignee: HD Inspiration Holding B.V., Emmeloord (NL)

(21) Appl. No.: 12/732,294

(22) Filed: Mar. 26, 2010

Related U.S. Application Data
(63) Continuation of application No. PCT/GB2008/050862, filed on Sep. 25, 2008.

(30) Foreign Application Priority Data
Sep. 27, 2007 (GB) 0718830.3

(51) Int. Cl. A61F 9/00; B29C 45/14

(52) U.S. Cl. 2/15; 264/271.1; 425/129.1

(57) ABSTRACT

There is provided a method of forming a visor having a spacer or seal extending along at least a portion of a periphery of a viewing area of the visor, comprising a step of injection molding the seal or spacer in a mold.

### CROSS REFERENCE TO RELATED APPLICATIONS

This application is a continuation of international application number PCT/GB2008/050862 and claims priority from United Kingdom application number 0718830.3 filed on Sep. 27, 2007, the contents of which are hereby incorporated by reference in their entirety.

### BACKGROUND OF THE INVENTION

1. Field of the Invention

The invention relates generally to a method of manufacturing a visor, particularly an overlay-visor, such as is used in a visor-assembly comprising an overlay-visor and a shield-visor releasably attached to one another. The invention also relates to an apparatus for implementing the method of the invention; a visor, particularly an overlay-visor, obtained from the method of the invention; a visor assembly comprising a visor obtained from the method of the invention; and a kit of parts for constructing a visor assembly.

More particularly the visors and visor assemblies of the invention are for personal protection equipment for facial and eye protection. Examples of personal protection equipment include motorbike helmets, motorbike style helmets such as quad-bike, snowmobile, racing car and skiing helmets; heavy-duty protective visors, for example, ballistic face shields which may be used in riot helmets and visors for use by the emergency services; and goggles such as underwater diving goggles, motorcycle goggles or skiing goggles. Windows in vehicles and instrument covers exposed to the open air, and the like, can also make use of the technology according to the invention.

2. Description of the Related Art

Visor assemblies comprising a shield-visor with an overlay-visor releasably attached thereto by mechanical fastenings, are known.

In such visor assemblies the shield-visor is more substantial than the overlay-visor and acts as a shield. In the case of motorbike style helmets the shield-visor acts to protect a user's face from wind, rain, dirt and grit; and in the case of ballistic visors it acts to protect the user's face from more substantial projectiles and blows. In goggles the shield-visor tends to be limited to extending over the eyes and that part of face immediately adjacent the eyes. The shield-visor of the goggles may have different functions depending on usage. For example diving goggles are worn to aid underwater vision, motorcycle goggles are worn to protect a user's eyes from projectiles and dirt, and ballistic goggles are worn to protect a user's eyes from more substantial projectiles. Goggles may be provided with two shield-visors, one per eye.

Shield-visors may be provided in 1-dimensional (1-D) form, that is occupying a single plane so as to be flat; 2-dimensional (2-D) form, that is curved in one direction; or 3-dimensional (3-D) form, that is curved in two directions so as to be bowled. Overlay-visors may be provided in appropriate shapes to fit to the surfaces of these various shield-visor forms. In this respect, overlay-visors may also be 1-D, 2-D or 3-D. 1-D overlay-visors are used with 1-D shield-visors; 1-D and 2-D overlay-visors are used with 2-D shield-visors; and 3-D overlay-visors are used with 3-D shield-visors.

The overlay-visor is typically utilized to provide an improved viewing window for the visor wearer. For example, the overlay-visor may be adapted to have an anti-condensation function to prevent misting-up of the viewing area. The overlay-visor may also or alternatively be provided with tinting to give improved viewing in varying light conditions. The viewing area of the shield-visor and/or the overlay-visor is the area through which the user looks.

Examples of helmet visor assemblies are known from U.S. Pat. Nos. 5,765,235 and 6,922,850, the contents of which are hereby incorporated by reference in their entirety, which provide anti-condensation overlay-visors attached to shield-visors.

In U.S. Pat. No. 6,922,850, prevention of misting-up of the viewing area is preferably achieved by provision of a chamber between an inner overlay-visor and a shield-visor. The chamber is filled with air or gas and acts to thermally insulate the internal surface of the overlay-visor from the external environment. The chamber is created by the provision of a flexible seal adhered to the overlay-visor and fitted detachably against the shield-visor so that the seal forms the peripheral boundary of the chamber. For the best anti-condensation results the chamber is sealed as far as possible with respect to the environment to prevent ingress of moisture and dirt to the chamber. In the preferred embodiment of U.S. Pat. No. 6,922,850 the seal is formed from a bead of silicone material adhered to the overlay-visor.

In a visor that is provided with a seal the viewing area is defined by the seal, which forms the boundary of the viewing area.

In order that the overlay-visor is detachable from the shield-visor, the silicone material is dry and set before it is brought into contact with the shield-visor. In this manner there is no adherence between the shield-visor and the spacer or seal so that the overlay-visor can be removed from the assembly or be replaced.

The current method of forming the overlay-visor with seal comprises applying a viscous silicone resin as an extruded bead of material along the periphery of a prefabricated overlay-visor using a computer numerical controlled (CNC) machine. The CNC machine controls a silicone dispensing nozzle which dispenses the bead of silicone resin under pressure onto the overlay-visor in the appropriate pattern. Once the resin has been applied to the overlay-visor, the bead dries/sets for between 24 to 48 hours depending upon the thickness of the spacer or seal and environmental conditions.

In such a process extremely accurate computer numerical control has to be utilised in order to achieve excellent bead placement, bead thickness and bead cross-section control. It has been identified by the inventor of the present invention that accurate control of the bead placement, bead thickness and bead cross-section are all essential to achieving a good seal to the chamber and hence a good anti-misting function of the overlay-visor. In order to achieve a highly effective seal, the dispenser speed, pressure, start and finishing points, and height of nozzle above the overlay-visor, all have to be synchronously coordinated. With such a process a number of problems occur.

The overlap at the start and finishing points for laying down the bead can cause problems with regards to optical quality of the final product as well as causing leakages into a sealed chamber because of a thickening or thinning of the bead at these points. The withdrawing of the nozzle can also lead to fowling of the nozzle.

The thickness and height of the completed set silicone bead cannot be guaranteed because the bead of silicone resin sets and flows differently according to the environmental conditions.

The viscosity of the silicone resin supplied to the CNC machine can vary between batches. This means that the thickness of the bead may vary depending on the batch of silicone resin. This may lead to overly thick or thin seals or spacers. Alternatively the viscosity of each batch of silicone resin must be tested and the CNC machine reprogrammed appropriately.

Once the silicone has been applied to the overlay-visor, it must be allowed to set in a controlled and dust free atmosphere for between 24 to 48 hours. If the drying room is not a clean room free of dust particles, these particles will irremovably ingress to the unset silicone resin. This leads to a poor optical quality of the set silicone and potentially poor sealing qualities.

For the drying process, costly covered stacking trays are needed to separate the sealed overlay-visors from each other horizontally and vertically.

Also in such a manufacturing process, great care has to be taken by the operator not to contact the unset silicone bead when handling the overlay-visor, in particular when removing the overlay-visor from the CNC machine. Any contact with the unset silicone bead will render the overlay-visor useless.

For these reasons the current manufacturing process is overly time consuming and too often produces a less than perfect spacer or seal.

It is the aim of the invention to overcome one or more of the above problems.

### BRIEF SUMMARY OF THE INVENTION

According to the present invention there is provided a method of forming a visor having a spacer or seal extending along at least a portion of a periphery of a viewing area of the visor, comprising a step of injection molding the seal or spacer in a mold.

Injection molding a seal or spacer directly onto a visor advantageously provides a visor having a seal or spacer adhered thereto, the method allowing a particularly accurate placement, sizing and/or shaping of the seal or spacer.

The method preferably comprises the steps of providing a visor; and injection molding an elastomeric seal or spacer onto at least portion of a periphery of a viewing area of the visor.

In one embodiment the method preferably comprises the steps of providing a mold positioned around the visor to produce a seal or spacer at the periphery of the viewing area; injecting an elastomer resin into the mold; and removing the visor from the mold.

In a more preferred embodiment there is provided a method of forming a visor provided with a spacer or seal extending along at least a portion of a periphery of a viewing area of the visor, comprising the steps of;

i) providing a mold comprising a first cavity shaped as the inverse of the visor and a second cavity shaped as the inverse of the spacer or seal and positioned in relation to the first cavity to produce a seal or spacer at the periphery of the viewing area;

ii) providing a visor in the first cavity;

iii) injecting an elastomer resin into the second cavity; and

iv) removing the visor provided with spacer or seal from the mold.

In the above embodiments the elastomer resin is preferably set before removal from the mold. However, in some circumstances it may be removed while the elastomer resin is still unset or only partially set.

The visor on which the seal or spacer is formed may be a shield-visor, however it is most preferably an overlay-visor. Provision on the overlay-visor allows retrofitting of an overlay-visor to a shield-visor and means that when a seal or spacer is damaged it is the less expensive overlay-visor which must be replaced and not the shield-visor.

A major advantage of the inventive method is found in that the sizing and location of the spacer or seal on the visor is greatly more accurate with such a method than with the prior method. Very accurate control of the spacer or seal has been found by the inventor to be an important feature in producing a high quality visor assembly. This is because a lack of accuracy when producing the spacer or seal can result in overly thick or thin spacers which in turn leads to sub-optimal spacing of the overlay-visor and the shield-visor and to poor sealing of a chamber between the overlay-visor and the shield-visor. This in turn leads to more frequent misting of the viewing area of the visor assembly and to a dirtying of the internal surfaces of the chamber.

Accurate sizing and placement of the spacer or seal is particularly important when an airtight seal is to be provided. This is even more especially the case when the overlay-visor is located on the outer-surface of for example the shield-visor of a motorbike helmet. In such circumstances the seal must prevent ingress of water, moisture and dirt under driving conditions, when the overlay-visor is subjected to extreme wind pressures at high speeds.

With the method of the present invention the seal or spacer size, and hence the distance between the overlay-visor and the shield-visor in a visor assembly, can be accurately and reliably controlled to optimise the anti-misting properties of the assembly. In the previous method such control was not possible leading to a sub-optimal spacing.

The use of an insulating air gap between the shield-visor and the overlay-visor provides good anti-misting properties without the need for electric heating elements as has been suggested in the prior art. The provision of electric heating elements is disadvantageous and it is hence preferred that the visor assembly of the present invention lacks electric heating elements.

It will be clear to those skilled in the art that the seal member may be provided at various locations on the overlay-visor or shield-visor so long as it encompasses an adequate viewing area for the visor user. For example the overlay-visor may be larger than the viewing area of the visor but the seal member located on the periphery of the viewing area, and thus not on the periphery of the overlay-visor.

The seal or spacer is formed from an elastomer resin. Examples of suitable elastomer resins for use in forming the spacer or seal include resins selected from the group consisting of silicone resins, epoxy resins and polyurethane resins. These may be thermoset or thermoplastic resins.

Preferably the resin is a thermoplastic silicone copolymer resin, which most preferably comprises at least 90% by weight of siloxane. Particular examples of such resins are Geniomer® 60 and Geniomer® 80 manufactured by Wacker-Chemie AG of Munich, Germany.

As an aid to improving the flow properties of the silicone resins, silica fume may be dispersed therethrough at a concentration of up to about 100 ppm.

The elastomer resin used to form the spacer or seal is preferably a low-temperature setting resin. This advantageously allows the use of low mold temperatures. High mold temperatures may damage the optical qualities of the visor; cause undesired size or shape changes in the visor; or adversely affect surface coatings or finishes (e.g. anti-scratch or anti-misting coatings) on the visor. For example, preferred materials for an overlay-visor are cellulose acetate and cellulose propionate. An overlay-visor made of these materials may suffer damage at temperatures above 89° C. and 98° C. respectively.

The elastomeric resin is preferably selected to be usable with a mold temperature of 90° C. or below, more preferably 70° C. or below; even more preferably 60° C. or below; and most preferably 50° C. or below, such as in a range of 20 to 50° C.

It is also possible to use the described silicone, epoxy and polyurethane resins in the manufacture of the visors by a method involving extrusion of a bead of the material from a nozzle onto the overlay-visor, as is the case in the already described prior method of manufacturing sealed overlay-visors.

The step of injecting the elastomer resin is preferably carried out with a screw speed of 100 to 200 rpm and with a back pressure of 0.1 to 1 MPa.

Another particular advantage of the use of injection molding to form the seal or spacer is found in the ability to form more complex structures than previously possible. The CNC dispensing method was only able to produce cross-sections similar to a spreading water droplet.

For example, in order to ensure a good sealing of the insulating chamber, the method may be adapted to produce a seal with two or more side by side ridges. These ridges may provide an improved sealing when biased against a visor surface of a visor assembly. The ridges may be substantially of the same height or one may extend further than the other. The production of the ridges is achieved by adapting the shape of the mold to the appropriate inverse of the desired seal or spacer shape.

Another example of a complex seal shape that can be formed by the inventive method is a seal or spacer that is adapted to interact with a mechanical fastening element on the shield-visor to which the overlay-visor is to be attached. In this manner the releasable mechanical fastening elements of the visor assembly can be formed as part of the seal. For example one of two interlocking components can be provided on the overlay-visor and the complementary component can be provided on the shield-visor. One example of such an arrangement is an interlocking groove and ridge fastener; commonly known as a ziplock fastener or a grip fastener. Such fasteners may be provided so as to be fastened and released by direct finger force or may be provided with a slider which runs along the groove and ridge fastener to fasten or release the join.

Another example of a seal obtainable via the injection molding method is one which varies in height along its length. This can be useful in the instance where it is desirable for a visor assembly to have a varied spacing of the overlay-visor and the shield-visor. For example, in a motorbike helmet visor assembly it may be desirable to have a narrow spacing at the upper portion of the visor assembly and a deeper spacing at the lower portion of the visor assembly. This helps to avoid a scratching contact of the overlay-visor with the helmet components when lifting the visor assembly. In another embodiment the spacing may be greater at the side portions than in the central portion.

In a particular example a seal may be provided which is about 0.5 mm in height along a first portion of a visor and is seamlessly graduated to 1 mm at an opposed portion of the visor. In this manner a sealed chamber is provided with a gradually increasing spacing from one portion to another.

The invention also relates to a visor, preferably an overlay-visor, provided with a spacer of seal having two or more ridges for contacting the surface of another visor; to a visor, preferably an overlay-visor, having a spacer or seal which is adapted to interact with a mechanical fastening element on another visor; and/or a visor, preferably an overlay-visor, having a spacer or seal varying in height to provide a varying spacing between an overlay-visor and a shield visor; independently of their method of production.

The mold of the invention is in the first instance shaped and positioned to produce a seal or spacer on only a first side of a visor. In other embodiments the mold may also be shaped and positioned to produce a seal or spacer on both a first and second surface of the visor. Providing the seal on both first and second surfaces of a visor allows the overlay-visor to be reversible and also to be placed optionally on the outside or inside surface of a shield-visor.

The mold may also be shaped and positioned to produce a seal or spacer on a portion of the edge of the overlay-visor. Most preferably the mold is adapted to produce a seal or spacer on a first and a second surface and on an edge of the overlay-visor.

A further advantage of the invention is found in that quick setting times in the range of less than 10 minutes, preferably less than 1 minute, more preferably less than 20 seconds and most preferably less than 10 seconds, can be achieved. This means that no extended setting times are required as was the case with the prior method. It also means that specialized drying rooms and racks are not required, and that the problem of operators contacting unset beads of resin is overcome.

The overlay-visor is in one embodiment provided as a visor prefabricated by cutting or milling from a sheet of material. Such a prefabricated visor may have been thermoformed into a 2-D or 3-D visor.

In another embodiment, in addition to injection molding the seal or spacer, the visor itself may be formed by injection molding. Injection molding of the visor may take place in the same mold in which the seal or spacer is injection molded in a multi-component molding process, usefully a two-component (2K) molding process. Alternatively the visor may be injection molded in a first mold and then have the seal or spacer added to it in a second mold.

It is most advantageous that the injection molding of both the visor and the seal or spacer be carried out in a single mold by multi-component injection molding, preferably a two-component (2K) molding technique. In 2K molding techniques three mold parts are used. Two parts of the mold, which produce a cavity the inverse of the first component, are used in the first stage of the process. Once the first component has been molded, one of the parts is removed and replaced with a third mold part. This third mold part contains an additional cavity that is the inverse of the second component.

In a two or multi-component method it is preferable that the first injection molded component is the visor and the second injection molded component is the seal. However, it is also possible to form the seal or spacer as the first injection molded component and to form the visor as the second injection molded component.

In some cases, for example where the seal or spacer is formed on both sides and the edge of the overlay-visor, a dual mold system may be used. A first mold is provided for forming the first component and the and a second mold is provided for adding the second component to the first component.

It is believed that injection molding of the overlay-visor produces an overlay-visor with an increased light transmission of approximately 1 to 2% compared to overlay-visors cut or milled from extruded sheets. This is important in ensuring that the visor assemblies of the invention meet the various governmental light transmission safety requirements. The increased optical quality is achieved because the molds are polished and optically corrected under computer control.

A multi-component or 2K injection molding process, similar to that described above, may be used to produce a shield-visor with a spacer or seal.

Further, in some instances it may be appropriate to make use of a 3 or more component injection molding process. For example, where the visor is itself injection molded and provided with seals or spacers on two sides, each of the seals or spacers may be formed from different materials. In such a situation a 3K injection molding process can be used.

A further aspect of the invention relates to an apparatus for implementing the above-described method. According to this aspect there is provided a mold for forming a visor, preferably an overlay-visor, provided with a spacer or seal extending along at least a portion of a periphery of a viewing area of the visor, the mold comprising a cavity shaped as the inverse of the spacer or seal and adapted to be positioned in use, in relation to the visor to produce a seal or spacer at the periphery of the viewing area.

As discussed above, the cavity of the mold may be adapted in various ways to produce a variety of seal or spacer shapes including complex shapes.

According to another aspect of the invention there is provided an overlay-visor adapted to be releasably attached to a shield-visor, comprising an injection molded spacer or seal extending along at least a portion of a periphery of a viewing area of the overlay-visor.

In line with this aspect there is also provided a visor assembly, comprising a shield-visor having a surface; an overlay-visor as discussed; and a mechanical fastening for releasably attaching the overlay-visor to the surface of the shield-visor. Preferably the assembly comprises a seal spacing the overlay-visor from the shield-visor and forming a sealed chamber between the shield-visor and the overlay-visor.

Further there is provided a kit of parts comprising an overlay-visor as discussed; and a shield-visor.

The overlay-visor in any of the above embodiments may be provided with any of the preferred spacer or seal shapes and placements.

In particular there may be provided an overlay-visor adapted to be releasably attached to a shield-visor, comprising a spacer or seal extending along at least a portion of a periphery of a viewing area of the overlay-visor, the seal or spacer comprising two or more ridges for contacting a surface of a shield-visor; and/or the seal or spacer being shaped to connect to a mechanical fastening element on a shield-visor.

Also there may be provided an overlay-visor adapted to be releasably attached to a shield-visor, comprising a spacer or seal extending along at least a portion of a periphery of a viewing area of the overlay-visor, the seal or spacer being provided on an edge of the overlay-visor.

In a preferred embodiment the shield-visor of the visor assembly is provided with a recess shaped to receive an overlay-visor. The dimensions of the recess preferably correspond closely to the peripheral dimensions of the overlay-visor. The depth of the recess is preferably such that when the overlay-visor is inserted it sits substantially flush with the un-recessed part of the shield-visor. In such an embodiment securing means may be provided in the form of a snap-fit rim or the like around at least a part of the recess periphery.

The provision of the seal or spacer on the edge of the overlay-visor is advantageous in the assembly where the shield-visor is provided with a recess for the overlay-visor. The edge located, elastomeric spacer or seal abuts the sidewalls of the recess fitting the overlay-visor snugly therein so as to reduce or prevent movement of the overlay-visor. Even in the event that some shrinkage should occur in the overlay-visor the compressed elastomeric seal or spacer located along its edge will take up the created space and maintain a firm retention of the overlay-visor.

As discussed, the overlay-visor maintains a user's vision through the visor assembly. In relation to this the overlay-visor is preferably provided with an anti-misting surface, for example, in the form of a surface having hydrophilic properties. The surface may be applied as a coating of a hydrophilic material. The coating is preferably a silicone based material which is applied by dip-coating. More preferably the overlay-visor is also provided with an anti-misting surface on both of its major surfaces.

One advantage of providing the coating on both surfaces is found in that a single coated overlay-visor can be located on either the inside of the outside surface of a 1-D or 2-D shield-visor. Since the overlay-visor is coated on both sides, a hydrophilic surface is presented on the outer surface of the overlay-visor in whichever position it is placed. Hence a single overlay-visor can provide the hydrophilic anti-misting function whether placed on the inner or outer surface of a shield-visor.

The overlay-visor may also be provided with a colouring agent in the form of a permanent colouring or a photochromic dye, such as a photo-chromic UV reactive dye.

This acts to reduce the ingress of excess light during, for example, sunny conditions, or to filter particular wavelengths of light.

The photo-chromic overlay-visor may take the form of a 3 ply laminate comprising two layers of polycarbonate and a central lamination adhesive impregnated with a powdered photo chromic dye. The percentage content of the dye can be varied as required in order to alter the level of shading of the overlay-visor in the activated state.

The overlay-visor may be provided with an anti-scratch coating, separately or in combination with an anti-misting surface, on either or both of it major surfaces. Preferably the overlay-visor is provided with an anti-misting surface on one side and an anti-scratch surface on the other side.

For curved shield-visors (2D and 3D), the overlay-visor may be provided on the inner concave surface of the shield-visor or on the outer convex surface of the shield-visor in order to prevent condensation formation on either the outside or the inside surface of the shield-visor. In a particular embodiment the shield-visor may be provided with both an inner overlay-visor and an outer overlay-visor, for environments where misting of both inner and outer shield-visor surfaces is likely to occur. This is particularly advantageous in the case where work in enclosed humid spaces may be expected. For example a ballistic visor, such as those worn by riot police or armed forces, may become misted on both outer and inner surfaces while waiting in a vehicle for orders to advance, or on entering a warm building after having been waiting in a cold environment.

In one embodiment of the invention the overlay-visor is adapted so that it can be fitted to both the inner surface and the outer surface of a shield-visor, for example on 1-D and 2-D shield-visors. This advantageously means that a single type of overlay-visor can be used as either an outer-overlay-visor or an inner overlay-visor. In an advantageous embodiment the overlay-visor may be provided with fastening portions on both of its major surfaces so that it can be fitted to either the inner or outer surface of a shield-visor. In addition the overlay-visor may be provided with spacer elements or seal elements on both of its major surfaces so that a single overlay-visor forms an air-gap whether placed on the outer surface or inner surface of a shield-visor.

In a preferred embodiment of the invention the overlay-visor is adapted to be fitted to the inner-surface of the shield-visor. Such an adaptation may, for example, be a shaping of the overlay-visor to fit the inner-surface of a recessed shield visor, the inner-surface of a 2-D shield-visor or the inner-surface of a 3-D shield-visor. More preferably the inner-surface of the shield-visor is provided with mechanical fastening elements for releasably attaching the overlay-visor thereto. A preferred embodiment of the visor assembly is one where the overlay-visor is provided on the inner-surface of the shield-visor.

According to a further aspect of the invention there is provided a helmet comprising; an opaque skull protection portion; and a visor assembly as discussed above.

The main body of the overlay-visor can be formed of a number of different materials, particularly preferred are polymeric resins. Examples of particularly preferred materials are cellulose propionate and cellulose acetate.

### BRIEF DESCRIPTION OF THE DRAWINGS

Specific embodiments of the invention will now be described by way of non-limiting example only. The features and advantages of the invention will be further appreciated upon reference to the following drawings, in which:

FIG. 1 shows a motorcycle style helmet provided with a visor assembly;

FIGS. 2, 2a and 2b show details of the visor assembly of FIG. 1;

FIGS. 3a to 3e show partial sections through overlay-visors with various spacer or seal forms;

FIGS. 4, 4a and 4b show a visor assembly having a shield-visor with a recess;

FIGS. 5a to 5d show a mold for injection molding of a seal;

FIGS. 6a and 6b show overlay visors with seals;

FIGS. 7a and 7b show a visor assembly having a ridge and groove attachment mechanism.

### DESCRIPTION OF ILLUSTRATIVE EMBODIMENTS

FIG. 1 shows a motorcycle helmet having an opaque skull protecting portion 3 to which is attached a 3D visor assembly 1. There is provided a shield-visor 2 having releasably attached to its inner-surface an overlay-visor 6. The overlay-visor 6 is releasably attached to the shield-visor 2 by mechanical fastenings 11 at opposed ends of the shield-visor 2.

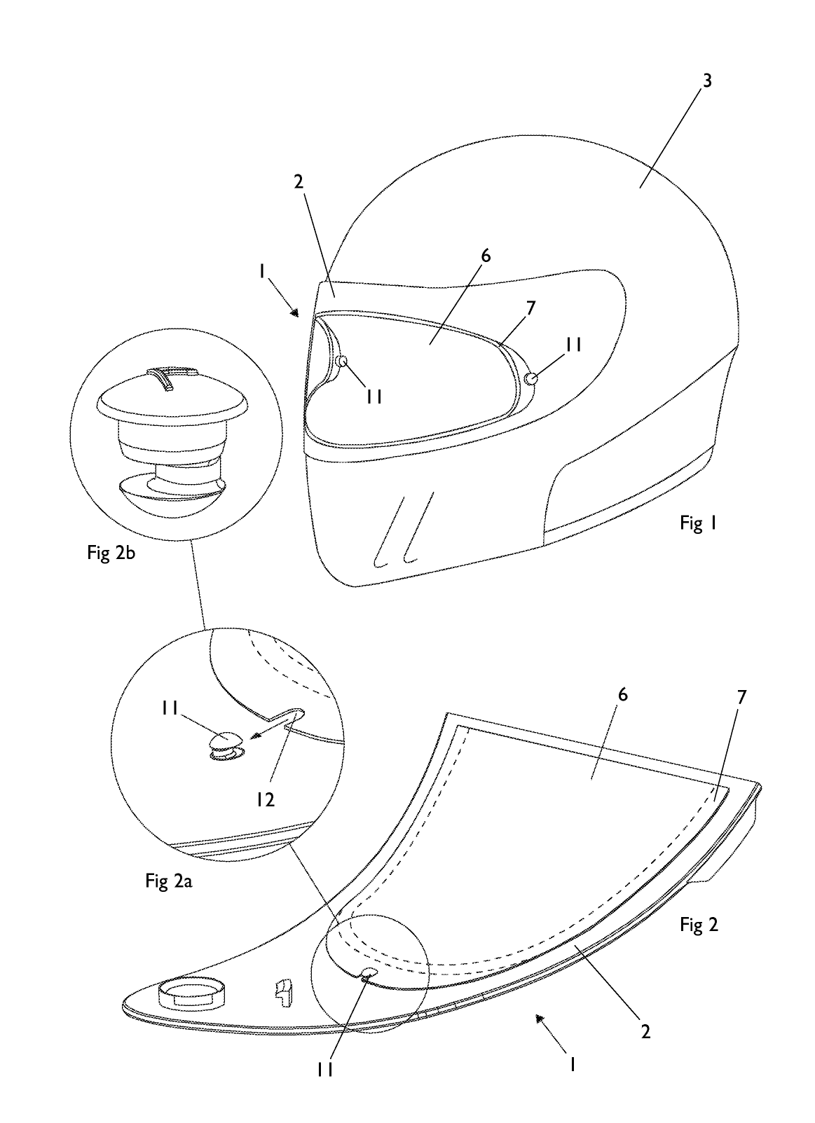

One of the mechanical fastenings 11 can be seen more clearly in FIGS. 2, 2a and 2b. It is comprised of an eccentric pin 11 fitted to the shield-visor 2. The overlay-visor 6 is provided with a recess 12 which mates with the eccentric pin 11. The mechanical fastenings 11 hold the overlay-visor 6 under tension within the inner curve of the shield-visor 2. The eccentric pins 11 of this embodiment are rotatable into and out of engagement with the recesses 12 of the overlay-visor 6 in order to ensure a secure retention thereof. In the event that the overlay-visor 6 should reduce in size in relation to the shield-visor 2, the eccentric pins 11 can be tightened to re-secure the overlay-visor.

Also illustrated in FIGS. 1 and 2 is a seal member 7 provided around the periphery of the overlay-visor 6. As a result of the presence of this seal the overlay-visor 6 is spaced from the shield-visor 2 and a chamber, sealed as far as possible with respect to the environment, is formed between the overlay-visor 6 and the shield-visor 2. This sealed chamber acts as an insulator reducing the possibility of condensation formation in the viewing area of the shield-visor 2.

The seal member 7 is adhered to the overlay-visor 6 and is held in non-adhesive relation to the shield-visor 2 by the mechanical fastening 11 so that it forms an airtight seal but does not adhere to the shield-visor 2. In this manner the overlay-visor 6 is removable from the shield-visor 2 so that it can be replaced if damaged, or removed or replaced depending upon weather conditions.

In one embodiment (not shown) the seal 7 may overlap slightly into the recess 12 so as to provide a resilient cushion abutting mechanical retaining pins.

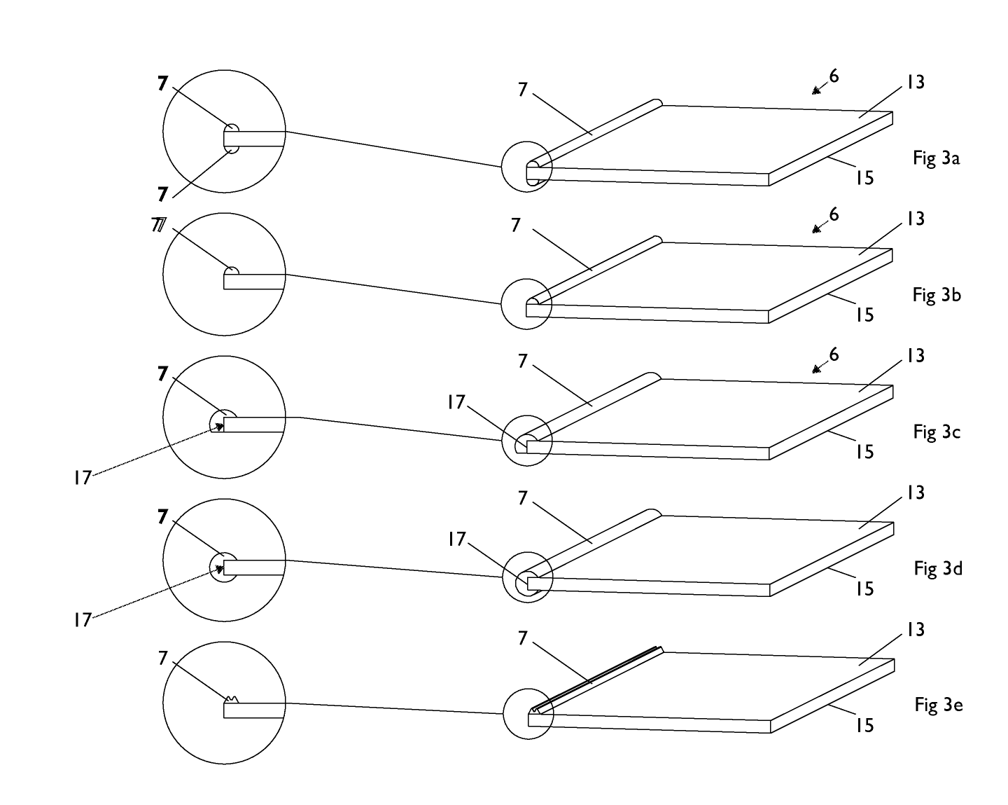

FIGS. 3a to 3d show partial sections through overlay-visors 6 provided with seal members 7.

In FIG. 3a the seal member 7 is provided on both a first surface 13 and a second surface 15 of the overlay-visor 6.

In FIG. 3b the seal member 7 is applied to only a first surface 13 of the overlay-visor 6.

In FIG. 3c the seal member 7 is provided on both a first surface 13 and the edge 17 of the overlay-visor 6.

In FIG. 3d the seal member 7 is provided on the first surface 13 and the second surface 15 as well as on the edge 17 of the overlay-visor 6.

In FIG. 5e the seal member 7 is provided on the first surface 13 of the overlay-visor 6 and is formed with a double ridge construction. Such a double ridge may be useful in providing an improved sealing of the chamber.

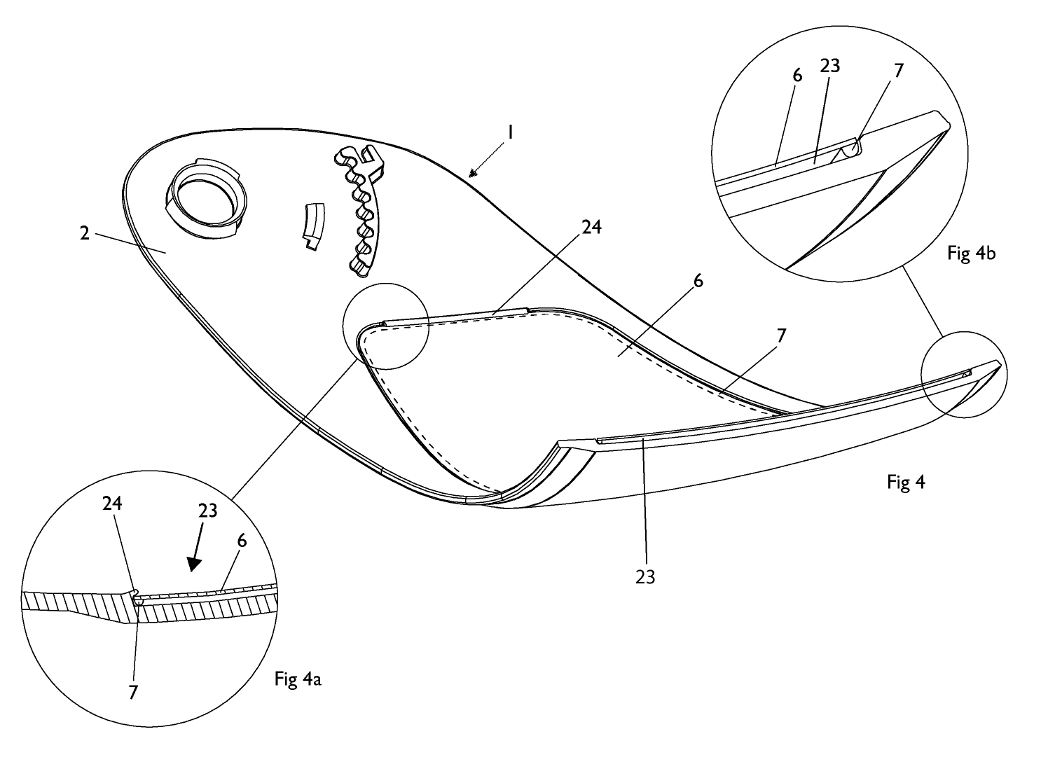

In FIGS. 4 and 4a there is shown a visor assembly 1 in which the shield-visor 2 is provided with a recess 23. The dimensions of the recess correspond to the external dimensions of the overlay-visor 6. The fastening in this embodiment is achieved by a snap-fit construction comprising snap-lips 24. This pushes the overlay-visor 6 against the shield-visor 2 with some pretension. Seal member 7 provides a seal between the overlay-visor 6 and the shield-visor 2, as a result of which ingress of moisture, and consequently misting up of the shield-visor 2, can be avoided.

The overlay-visors can be manufactured by a process in which the seal 7 is injection molded directly onto the overlay-visor 6. In such a process use is made of a mold 30. Partial cross-sections of a mold are shown in FIGS. 5a to 5d. The mold has a first cavity shaped to receive a prefabricated overlay-visor 6. The mold is also provided with a second cavity which as can be seen in the figure is shaped as the inverse of the desired seal shape 7, and is placed in relation to the first cavity such that the seal is formed on the periphery of the viewing area of the overlay-visor 6. The mold shown in FIGS. 5a to 5d is adapted to provide a seal on both surfaces and the edge of the overlay-visor.

In the method a prefabricated overlay-visor is placed into the first cavity of the mold and the mold is closed as in FIGS. 5a and 5b. Geniomer® 60 or 80, produced by Wacker-Chemie AG of Munich Hamburg, which is a low temperature, quick-setting, thermoplastic silicone resin having greater than 90% siloxane content, is then injected into the second cavity of the mold; FIG. 5c. The mold is held at a temperature of 20-50° C. for the injection step.

Following the injection step the injection resin is allowed to set for 10 to 20 seconds and then the overlay-visor with seal or spacer is removed from the mold; FIG. 5d.

For Geniomer® 60 the following injection molding conditions are used:

| Melt Temperature | 100-170° C. | |
|---|---|---|
| Barrel Temperatures | 1 | 100-120° C. |
| | 2 | 140-160° C. |
| | 3 | 140-160° C. |
| | Die | 140-160° C. |

| | |
|---|---|
| Mold Temperature | 20-50° C. |
| Screw speed (general purpose | 100-200 rpm |
| screw) | |
| Cooling time | 10-20 sec |
| Back Pressure | 0.1-1 MPa |

For Geniomer 80 the following injection molding conditions are used:

| Melt Temperature | 185-200° C. | |
|---|---|---|
| Barrel Temperatures | Rear | 100-170° C. |
| | Middle | 170-200° C. |
| | Front | 180-200° C. |
| | Nozzle | 185-200° C. |

| | |
|---|---|
| Mold Temperature | 20-50° C. |
| Screw speed (general purpose | 100-200 rpm |
| screw) | |
| Cooling time | 10-20 sec |
| Back Pressure | 0.1-1 MPa |

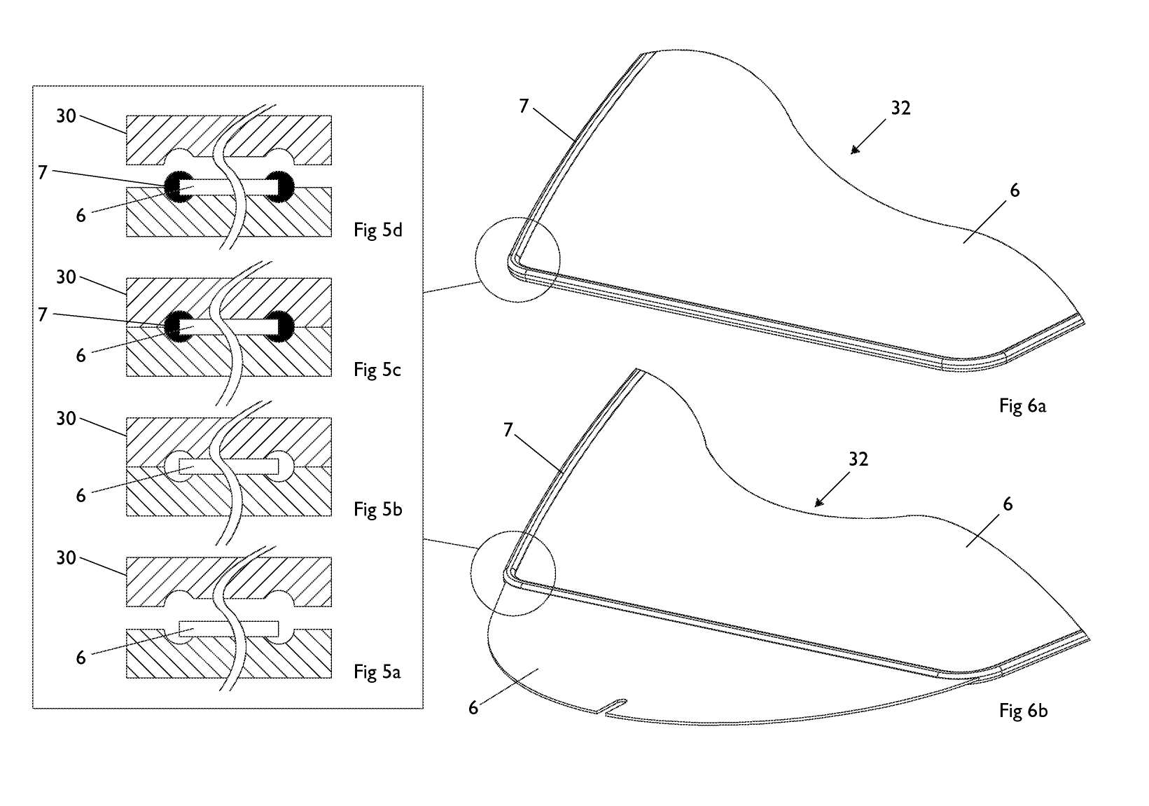

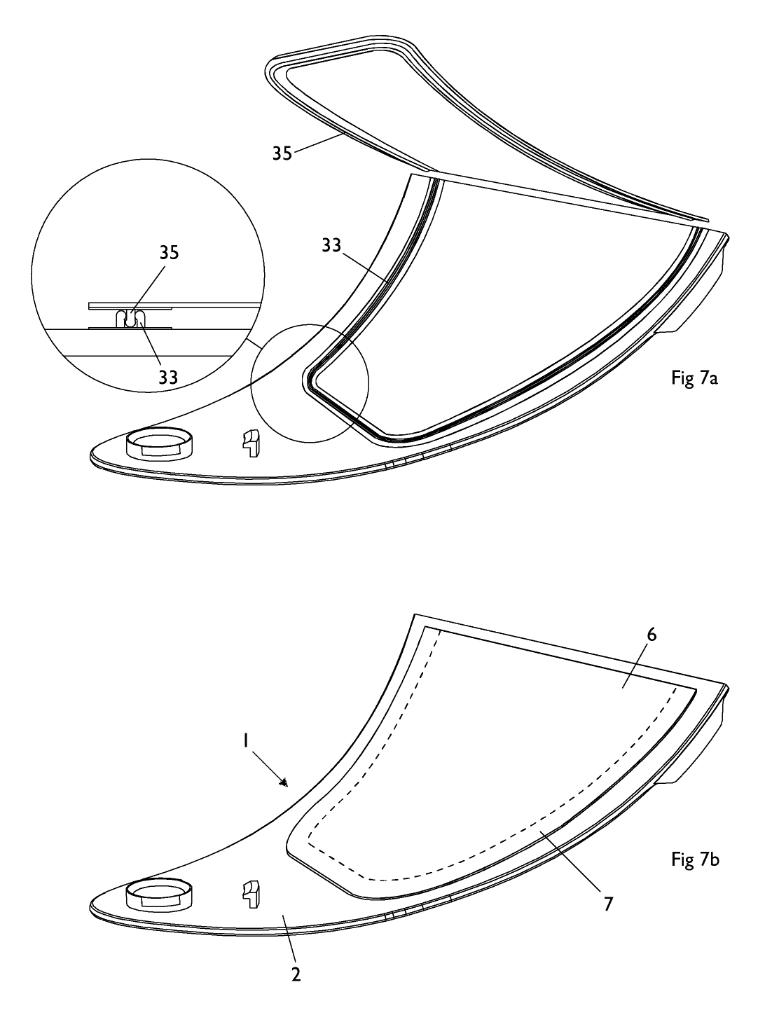

FIGS. 6a and 6b show examples of overlay-visors which may be produced from molds in accordance with FIGS. 5a to 5d. In FIG. 6a the seal 7 extends around the periphery of the overlay-visor 6 on both its surfaces 13, 15 and its edge 17. In FIG. 6b, the seal extends around the periphery of a viewing area 32, but not around the whole of the periphery the overlay-visor 6. In such an embodiment the second cavity in the mold 6 is shaped and located appropriately to provide such a seal 7.

The over-lay visor is provided as a prefabricated component and is either cut or milled from an extruded sheet; or is injection molded.

In an alternative embodiment (not shown) a multi-component injection molding process is used to form the overlay-visor. In this process the overlay-visor is injection molded into the same mold in which the seal is formed.

Alternative shapes to those shown in FIGS. 5a to 5d of the second cavity of the mold are used to provide alternative seal shapes and cross-sections, examples of which are shown in FIGS. 3a to 3d.

Preferably a coating, for example an anti-misting coating, is provided on a prefabricated the overlay-visor prior to insertion into the mold. However, it is also possible that a coating is provided on the overlay-visor (prefabricated or multi-component injection molded) after the seal or spacer has been applied. This is done by dipping the overlay-visor in a vat of the appropriate coating.

In an alternative embodiment (not shown) a seal 7 or spacer may be provided on the shield-visor rather than on the overlay-visor. Similarly to the overlay-visor the seal may be provided by injection molding onto a prefabricated shield-visor or by multi-component injection molding of the shield-visor and seal or spacer.

In such an embodiment an overlay-visor not provided with a seal or spacer is placed over the seal to create an insulating gap or chamber. Hence a similar anti-misting effect can be achieved as with the above described embodiments. However, this embodiment is less preferred because on suffering damage to the spacer or seal the shield-visor must be discarded. Since the shield-visor is typically more expensive than the overlay-visor, it is preferred that in the event of damage to the seal it is the overlay-visor that is discarded.

A preferred embodiment of the invention is shown in FIGS. 7a and 7b, in which an interlocking groove 33 and ridge 35 fastening system has been formed for releasably fastening the overlay-visor 6 to the shield visor 2. In this embodiment the seal 7 on the overlay-visor 6 is injection molded as a ridge 35 which mates with a groove shaped fastening element 33 on the shield visor 2. The groove shaped element 33 on the shield visor may be injection molded onto the shield-visor 2, may be adhered thereto as a preformed element, or may be integrally formed with the shield-visor 2. The ridge and the groove elements may be formed from the same or different materials as one another. In such an embodiment the overlay-visor is fastened to the shield-visor by pressing the ridge into the groove (FIG. 7b).

Many modifications in addition to those described above may be made to the structures and techniques described herein without departing from the spirit and scope of the invention. Accordingly, although specific embodiments have been described, these are examples only and are not limiting upon the scope of the invention.

It should be noted that the term “comprising” as used in the claims or description of this application does not exclude other elements or steps; and the terms “a” and “an” do not exclude a plurality.

Equivalents and modifications not described above may also be employed without departing from the scope of the invention, which is defined in the accompanying claims.

What is claimed is:

1. A method of forming a visor having a spacer or seal extending along at least a portion of a periphery of a viewing area of the visor, comprising a step of injection molding the seal or spacer in a mold.

2. The method according to claim 1, wherein the mold is positioned around the visor to produce the seal or spacer at the periphery of the viewing area; and wherein the method further comprises the steps of:
i) injecting an elastomer resin into the mold; and
ii) removing the visor provided with spacer or seal from the mold.

3. The method according to claim 2, wherein the elastomer resin is a low-temperature, quick-setting elastomer resin.

4. The method according to claim 2, wherein the elastomer resin comprises at least one of: silicone resins, epoxy resins and polyurethane resins.

5. The method according to claim 4 wherein the resin comprises a thermoplastic silicone copolymer resin.

6. The method according to claim 2, wherein the resin comprises silica fume dispersed therethrough.

7. The method according to claim 2, wherein at step i) a mold temperature is less than 90° C.

8. The method according to claim 1, wherein the mold is shaped to form the seal or spacer having two or more ridges for contacting a surface of another visor.

9. The method according to claim 1, wherein the mold is shaped to form the seal or spacer that is adapted to interact with a mechanical fastening element on another visor.

10. The method according to claim 1, wherein the mold is shaped and positioned to produce at least one of: the seal or spacer on both a first and second surface of the visor; the seal on a first or a second surface and on an edge of the visor; and the seal on a first and a second surface and on an edge of the visor.

11. The method according to claim 1, further comprising the step of injection molding the visor.

12. The method according to claim 1, wherein the visor is an overlay-visor.

13. The method according to claim 12, wherein the overlay-visor is adapted to be placed on an inner surface of a shield-visor.

14. A mold for forming a visor wherein the visor is provided with a spacer or seal extending along at least a portion of a periphery of a viewing area of the visor, the mold comprising a cavity shaped as the inverse of the spacer or seal and adapted to be positioned, in use, to produce the seal or spacer at the periphery of the viewing area.

15. The mold according to claim 14, wherein the cavity is shaped and adapted to be positioned, in use, to produce at least one of: the seal or spacer on both a first and second surface of the visor; the seal or spacer on a first or a second surface and on an edge of the visor; and to produce a seal or spacer on a first and a second surface and on an edge of the visor.

16. The mold according to claim 14, wherein the cavity is shaped to form the seal or spacer having two or more ridges for contacting a surface of another visor.

17. The mold according to claim 14, wherein the cavity is shaped to form the seal or spacer that is adapted to interact with a mechanical fastening element on another visor.

18. The mold according to claim 14, wherein the mold is adapted to form an overlay-visor.

19. The mold according to claim 18, wherein the overlay-visor is adapted to be placed on an inner surface of a shield visor.

20. An overlay-visor adapted to be releasably attached to a shield-visor, comprising an injection molded spacer or seal extending along at least a portion of a periphery of a viewing area of the overlay-visor.

21. The overlay-visor according to claim 20, wherein the spacer or the seal comprises two or more ridges for contacting a surface of the shield-visor.

22. The overlay-visor according to claim 21, wherein the spacer or the seal is shaped to interact with a mechanical fastening element on the shield-visor.

23. The overlay-visor according to claim 22, wherein the mechanical fastening element comprises an interlocking groove and ridge fastening mechanism.

24. The overlay-visor according to claim 20, wherein the seal or spacer is provided on: both a first and second surface of the overlay-visor; on a first or a second surface and on an edge of the overlay-visor; or on a first and a second surface and on an edge of the overlay-visor.

25. The overlay-visor according to claim 20, wherein the overlay-visor is provided with at least one of: a hydrophilic surface, an anti-scratch surface, and a photochromic shading.

26. The overlay-visor according to claim 20, wherein the overlay-visor is at least one of injection molded and adapted to be placed on an inner surface of the shield visor.

27. A visor assembly, comprising:
a shield-visor having a surface;
an overlay-visor adapted to be releasably attached to the shield-visor, comprising an injection molded spacer or seal extending along at least a portion of a periphery of a viewing area of the overlay-visor; and
a mechanical fastening for releasably attaching the overlay-visor to the surface of the shield-visor.

28. The assembly according to claim 27, wherein the seal spaces the overlay-visor from the shield-visor and forms a sealed chamber between the shield-visor and the overlay-visor.

29. The assembly according to claim 27, wherein the shield-visor has an outer-surface and an inner-surface; and the overlay-visor is releasably attached to the inner-surface of the shield-visor

30. The assembly according to claim 27, wherein the visor assembly is attached to a helmet comprising an opaque skull protection portion.
